(19) United States
(12) Patent Application Publication
(10) Pub. No.: US 2021/0252507 A1
(43) Pub. Date: Aug. 19, 2021

(54) APPARATUS FOR SEPARATING THE CELLULAR AND LIQUID PORTIONS OF A WHOLE BLOOD SAMPLE

(71) Applicant: UNIVERSITY OF MARYLAND, BALTIMORE COUNTY, Baltimore, MD (US)

(72) Inventors: Mustafa AL-ADHAMI, Baltimore, MD (US); Yordan KOSTOV, Baltimore, MD (US); Govind RAO, Baltimore, MD (US)

(21) Appl. No.: 17/176,422

(22) Filed: Feb. 16, 2021

Related U.S. Application Data
(60) Provisional application No. 62/977,368, filed on Feb. 16, 2020.

(51) Int. Cl. B01L 3/00; B01D 21/00; B01D 21/24; B01D 15/38

(52) U.S. Cl. CPC B01L 3/502715; B01D 21/0087; B01D 21/2444; B01L 2200/0647; B01D 15/3804; B01L 2300/0883; B01D 21/245

(57) ABSTRACT

A microfluidic device and method of using same to separate whole blood into at least a plasma serum fraction and a red blood cell-containing fraction. The microfluidic device uses the principle of sedimentation and the different densities of the cells relative to the plasma serum to separate whole blood into at least the two fractions.

### CROSS REFERENCE TO RELATED APPLICATIONS

This application claims priority to U.S. Provisional Patent Application No. 62/977,368 filed on Feb. 16, 2020 in the name of Mustafa Al-Adhami, et al., and entitled “Blood Plasma Separation in a Channel,” which is hereby incorporated by reference herein in its entirety.

### FIELD

The present invention relates to a microfluidic device and method of using same to separate blood into two or more fractions for collection of cells and other solid matter (for example, red blood cells, white blood cells, and plasma), plasma, or both cells and plasma.

### BACKGROUND OF THE INVENTION

It can be useful to separate blood plasma from whole blood, for example to facilitate analysis of one or more components of the blood plasma with minimal interference of the red blood cells. Alternatively, the invention can be used to isolate prokaryotic or eukaryotic cells from other solid matter of a blood sample.

### SUMMARY OF THE INVENTION

In one aspect, a microfluidic cassette is described, said microfluidic cassette comprising: a base comprising a microfluidic channel, wherein the microfluidic channel approximates a pattern selected from the group consisting of a substantially circular spiral, a substantially elliptical spiral, a substantially serpentine pattern, and a substantially straight channel,
wherein the microfluidic channel has a cross-section that is square, rectangular, circular, triangular, polygonal, or elliptical, and is about 0.5 mm to about 10 mm deep and about 0.5 mm to about 10 mm wide.

In another aspect, a method of separating whole blood into at least a fraction comprising plasma serum and a fraction comprising cells and other solid matter is described, said method comprising: introducing whole blood into a microfluidic channel of a microfluidic cassette described herein, holding the whole blood in the microfluidic channel for an amount of time necessary to effectuate substantial sedimentation, resulting in a top layer that comprises plasma serum and a bottom layer that comprises cells and other solid matter; and removing the layers by pumping or vacuuming, wherein the top layer moves with a higher velocity than the bottom layer, and the top layer comprising the plasma serum moves past the bottom layer to an exit port.

Other aspects, features and embodiments of the invention will be more fully apparent from the ensuing disclosure and appended claims.

### BRIEF DESCRIPTION OF THE FIGURES

FIG. 1A is a photograph of an embodiment of the microfluidic device described herein, complete with a Y-junction and a tube connected to an entry port of the channel of the device.

FIG. 1B is an example of a substantially circular spiral pattern.

FIG. 1C is an example of a substantially elliptical spiral pattern.

FIG. 2A is a schematic of the use of the microfluidic device to separate plasma serum from the cells and other solid matter of a blood sample.

FIG. 2B is a photograph of the plasma serum separated from the red blood cells in a channel

FIG. 2C is a photograph of the plasma serum separated from the red blood cells using the device and method described herein.

FIG. 3 is a photograph of the plasma serum separated from the red blood cells using the device and method described herein compared to plasma serum collected using centrifugation.

FIG. 4 is two embodiments of a device comprising two layers.

### DETAILED DESCRIPTION, AND PREFERRED EMBODIMENTS THEREOF

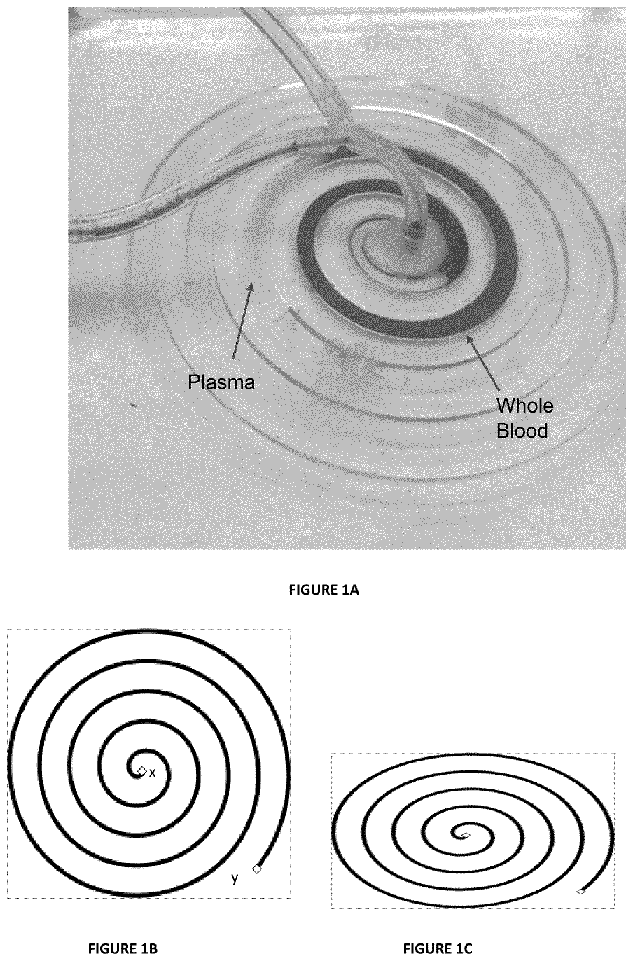

As defined herein, a substantially “circular spiral” pattern is understood to have a center point and a series of substantially circular shapes that orbit around the center point, moving farther away from the center point with each orbit. The circular spiral can approximate an Archimedean spiral, a hyperbolic spiral, a Fermat's spiral, a logarithmic spiral, or the like. An example of a substantially circular spiral pattern is shown in FIG. 1B.

As defined herein, a substantially “elliptical spiral” pattern is understood to have a center point and a series of substantially elliptical shapes that orbit around the center point, moving farther away from the center point with each orbit. An example of a substantially circular elliptical pattern is shown in FIG. 1C.

As defined herein, a “serpentine” pattern is understood to comprise a sinusoidal-like, winding pattern. The curves in the pattern can be smooth arcs or can be more square or angular.

As defined herein, “plasma” and “serum” are intended to be interchangeable terms. Often they are presented together as “plasma serum.”

As defined herein, “highly pure” plasma serum obtained using the device and method described herein corresponds to at least 95% purity, preferably at least 97%, even more preferably at least 99%, and most preferably at least 99.5% purity (i.e., less than 0.5% is cellular material or other solid matter).

As defined herein, the “cells and other solid matter” found in blood comprise, consist of, or consist essentially of, red blood cells (or erythrocytes), white blood cells (or leukocytes), and platelets (or thrombocytes), as well as fragments and subcellular components of any of the cells.

As used herein, “substantially” is intended to denote that the shapes or patterns described may not absolutely be square or circular, for example, but do approximate the shapes or patterns described.

“Substantially devoid” is defined herein to mean that none of the indicated substance is intentionally added to or present in the composition.

As defined herein, “substantial sedimentation” corresponds to at least 95% of the cells and other solid matter in the whole blood have settled, or are no longer suspended in the plasma serum, preferably at least 97%, more preferably at least 99% and most preferably at least 99.5%.

To date, there is no rapid and effective way to separate the cellular and liquid portions of a whole blood sample when a diagnostic test requires a sample of plasma serum. Traditionally, the cells have been removed using centrifugation, which relies on the cells and other solid matter having a higher density than the density of the serum, centrifugation being utilized as a means to speed up the process. Disadvantageously, centrifuging blood can damage at least a portion of the red blood cells, resulting in a possible leakage of hemoglobin therefrom, which can interfere with future spectroscopic assays. Furthermore, an amount of damaged red blood cells can remain in the plasma serum post-centrifugation, causing unstable spectroscopic readings. To avoid this, the leftover cells have been treated with lysing buffer, however, the buffer can interfere with spectroscopic assays as well. For example, if the bacteria in blood is being measured, lysing chemicals not only lyse red blood cells but also some of the bacteria, resulting in an inaccurate measurement. Finally, centrifugation is performed in a special vessel (e.g., a centrifuge tube), which typically requires handling by a trained technician. For example, the whole blood has to be transferred to a centrifuge tube and following centrifugation, the serum supernatant is aspirated, typically using pipettes. It is a difficult laboratory step for automation, and it is very problematic for interfacing with microfluidics, as performing it requires a break in the process flow. Moreover, sample sterility can be compromised using centrifugation.

The instant device and method eliminate the need for centrifugation to isolate cells and other solid matter from plasma serum, as well as eliminating the need to add chemicals to a sample to achieve cell lysis, for example. The instant device and method of using same relies on the different densities of the cells and other solid matter relative to the plasma serum to separate the two fractions.

Broadly, the instant invention relates to a device, such as a microfluidic device, and a method of using same to sediment the cells and other solid matter therein, yielding a sample of plasma serum for subsequent or future analysis. An embodiment of the microfluidic device in which the process occurs is shown in FIG. 1A. The device can be a microfluidic device having a microfluidic channel therein, wherein the channel can have, or approximates, a pattern selected from the group consisting of a substantially circular spiral, a substantially elliptical spiral, a substantially serpentine pattern, a substantially straight channel, or some other pattern that maximizes channel volume over a set area (e.g., a channel of a given length regardless of the pattern). Minor variations of the pattern are readily envisioned by the person skilled in the art, for example, minor variations are known in the heating and cooling field, e.g., in radiant floor heating, wherein the pipework is laid out in a substantially circular spiral, a substantially elliptical spiral, or a substantially serpentine pattern. In one embodiment, the entire channel resides on a single plane, i.e., there is no intended incline or decline in the channel at any point in the device. In another embodiment, the channel is multilayered, wherein each channel layer resides in its a dedicated plane, i.e., there is no intended incline or decline in the channel in that dedicated layer. Examples of a multi-layer channel are shown in FIG. 4. It should be appreciated that the channel within a plane may have slight imperfections, e.g., divets, etc., depending on how the device is manufactured. It should also be appreciated that FIG. 4 is not in any way intended to limit the device, which can have more than two layers and/or the multilayers can be arranged differently than illustrated in FIG. 4.

The cross-sections of the microfluidic channels can be substantially square, substantially rectangular, substantially circular, triangular, polygonal, or substantially elliptical. One such example is a microfluidic device comprising a microfluidic channel that is 3 mm deep and 3 mm wide, which can be a square channel or a circular channel having a diameter of 3 mm. For the purposes of this invention, the cross-section of the microfluidic channels can be in a range from about 0.5 mm to about 10 mm, preferably about 1 mm to about 5 mm, and more preferably about 2 mm to about 4 mm, deep, and about 0.5 mm to about 10 mm, preferably about 1 mm to about 5 mm, and more preferably about 2 mm to about 4 mm, wide. It should be appreciated that the depth and width (or diameter) of the channel can be the consistent throughout the device, or can have varied dimensions, as understood by the person skilled in the art.

The volume of blood that can be processed is determined by the combination of the length of the microfluidic channel, the pattern, and the cross-sectional dimensions of the channel For example, the length of the microfluidic channel having a 3 mm deep and 3 mm wide cross-section can be in a range from about 5 cm to about 100 cm, depending on the volume of processed fluid (e.g., blood). The person skilled in the art will understand how to adapt the length of the channel based on the cross-section dimensions.

The microfluidic device can comprise, for example, an acrylic base where the channel is cut. In addition to acrylic, other materials that can be used include, but are not limited to, polystyrene, polycarbonate, polyesters, celluloids (e.g., cellulose acetate or similar cellulosic derivatives), polydimethylsiloxane (PMDS), and any other thermoplastic or thermoset resins. Any 3D printable material can be used as well. The base has a depth of equal to the depth of the intended channel For example, for a channel having a depth of about 3 mm, the depth of the base is about 3 mm. The base is then covered on the top and on the bottom with a sheet cover of poly (methyl methacrylate) (PMMA) or polystyrene or tape of any kind to form a fully enclosed device. The sheet covers can be each about 0.2 mm to about 1 mm thick. It should be appreciated by the person skilled in the art that the microfluidic device can comprise only one layer if 3D printing is used or only two layers if the channels are engraved or molded and not cut through from the top to the bottom of the base (and only one sheet cover is used to cover the base). Alternatively, the base of the device can be manufactured using photolithographic techniques, as understood by the person skilled in the art. The chemical makeup of the microfluidic device is important. Specifically, the base, the sheet(s), and the “glue” that is used to attach the base to the PMMA or polystyrene sheet layers must not react with the blood sample. Preferably, adhesives are not used and the device is sealed by pressure, temperature, and/or weak solvent assistance. For example, the microfluidic device can be heat treated to bond the layers, as readily understood by the person skilled in the art.

Alternatively, the microfluidic device comprises a tube, for example, a tube having an inner diameter having the preferred cross-sectional channel dimensions, affixed to a surface/base. The tube can be affixed to the surface in a pattern selected from the group consisting of a substantially circular spiral, a substantially elliptical spiral, a substantially serpentine pattern, a substantially straight channel, or some other pattern that maximizes channel volume over a set area. The tube can comprise vinyl, TYGON, silicone, polyetheretherketone (PEEK) or some other polymeric material that is inert to blood. It is also contemplated that the tube not be affixed to a surface at all, e.g., when the tube is run substantially straight along a table or the like.

Similar to that shown in FIG. 1A, a tube can be connected to an entry port and a second tube can be connected to an exit port of the microfluidic channel of the device. For example, the entry port may be at or near the center point of a spiral (e.g., “x” in FIG. 1B) and the exit port can be where the spiral ends (e.g., “y” in FIG. 1B), or vice versa. Alternatively, the entry port can be at one end of the serpentine pattern and the exit port can be at the other end of the serpentine pattern (not shown). The connection of the tubes to the entry and exit ports of the channel of the device are understood by the person skilled in the art. In a preferred embodiment, the connectors can withstand pump pressures or vacuums without disconnecting. Examples of connectors include, but are not limited to, luer locks, luer slips, and barbed fittings. In a preferred embodiment, the tubes and the connections comprise a material that is inert to blood and any cleaning composition or diluent.

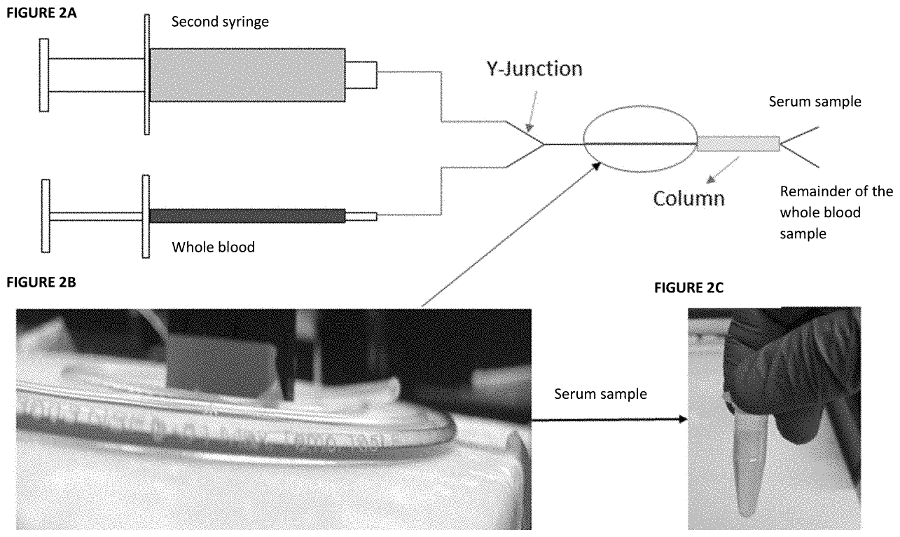

In practice, the blood is introduced into the microfluidic device at the entry port, for example, using a syringe, syringe pump, vacuum or other method or device that produces minimum shear stress on the cells (see, for example, FIG. 2A). The blood can be introduced to the device, either diluted or undiluted. The blood, diluted or not, is held in the channel of the microfluidic device to sediment. Depending on the specifications of the device and sample, e.g., cross-sectional dimensions of the channel, extent of dilution, etc., the sedimentation preferably takes about 15-60 minutes to complete. Notably, the lower the depth of the channel, the faster the sedimentation process. When the cells and other solid matter are fully sedimented and packed together, two distinct layers in the fluid are present, the serum on the top is yellowish and the cells and other solid matter on the bottom exist as a dense, bright red layer (see, FIG. 2B). As a result of the close packing, the two layers now have two distinct densities. The solution in the channel is then removed by pumping or vacuum, wherein the two different layers move with different velocities. As a result, the serum travels the fastest and moves past the cells in the channel towards the exit port. In one embodiment, there is a second syringe, having saline, buffer, or air therein, wherein the contents of the second syringe are used to elute the plasma serum out while the cells and other solid matter stay in the channel/tubing. Optionally, downstream of the device a column or some other affinity binding device can be included to improve purity, wherein the column comprises a resin (e.g., HISPUR Cobalt resin) to bind any residual hemoglobin/red blood cells, thereby further improving the purity of the plasma. The plasma serum can be collected as an initial fraction of the liquid exiting the microfluidic channel The later fractions comprising a fraction of mostly the cells can be collected as well. In one embodiment, the collected plasma serum is highly pure, having an extremely low concentration of cells and other solid matter in it and is substantially devoid of hemoglobin, lysing chemicals, and anti-coagulants. In another embodiment, the collected plasm serum comprises prokaryotic or eukaryotic cells because blood cells sediment much more quickly than the prokaryotic or eukaryotic cells. The prokaryotic or eukaryotic cells therefore can be suspended in the plasma serum after sedimentation and thereafter be removed with the plasma serum fraction.

It should be appreciated that in one embodiment, no column other affinity binding device is necessary and highly pure plasma serum can be obtained. Further, in another embodiment, the column other affinity binding device is used to obtain highly pure plasma serum.

It should be appreciated that the removal of the plasma serum (and then the cells and other solid matter) from the microfluidic channel can be performed mechanically, for example, using a pump or a vacuum, or can be performed manually, for example using the second syringe, as described herein. It should also be appreciated that the plasma serum can be pushed out of, or pulled out of, the microfluidic channel

The described device and method of using same is a purely microfluidic approach to serum separation. The plasma serum sample can be collected or directed to subsequent filtering, reaction, or monitoring assays. Advantageously, once the blood is introduced into the device, there is no need for handling by a technician to transfer it for further treatment, e.g., as is typical of a centrifuge, as the sedimentation and separation can be done within the device. This greatly reduces pumping and handling volume errors. Additionally, the fact that process is performed in a closed system results in improved sterility and overall safety by reducing the possible exposure of the technicians to biohazards.

Following separation of a sample in the microfluidic device, a syringe comprising a cleaning composition attached to the Y-junction can be used to flush the microfluidic device for reuse (see, e.g., FIG. 2A). It should be appreciated that the number of syringes attached to a Y-junction can be more than two, for example, 3, 4, 5, or more, depending on the number of liquids to be introduced to the microfluidic device at different moments in the separation or cleaning process. It should also be appreciated that the microfluidic device can be disposable as well.

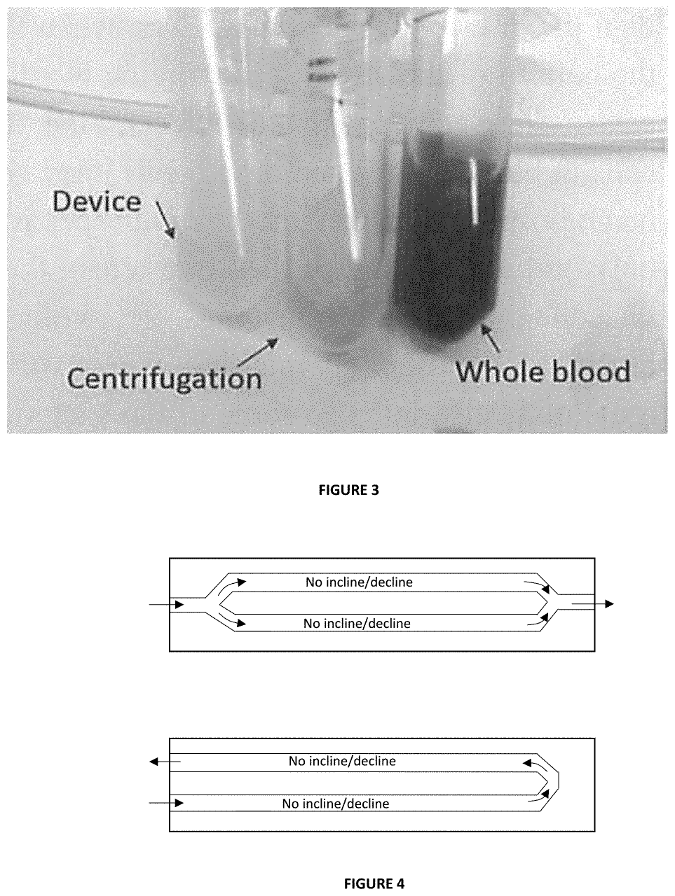

It can be seen in FIG. 3 that the plasma serum obtained using the device and the method of using same (yellow) is superior to that obtained using centrifugation (pink). It is clear that the latter still comprises a large number of red blood cells, possibly leaking hemoglobin, and lysing of the cells is likely necessary. The presence of hemoglobin and/or lysing chemicals will interfere with many downstream assays and is preferably avoided. Advantageously, the present invention device and method avoids the use of centrifuges or anything else requiring centrifugal force, filtration units (e.g., dead-end, cross-flow, etc.) or any other separation devices requiring membranes, lysing chemicals, and other chemicals such as anti-coagulants, by using the principle of sedimentation in a microfluidic device, providing a plasma serum sample comprising a low concentration of cells and no lysing chemicals, and doing so in a fraction of the time typically needed for centrifugation. Further, the device and method of using same is simpler to use than centrifugation and has improved sterility and overall safety by reducing the possible exposure of the technicians to biohazards. Further, because the separation occurs at a micro-scale, less patient blood is needed.

It should be appreciated that although the emphasis is on the obtainment of the plasma serum, the instant device and method of using same also provides for a rapid and efficient way to obtain a sample substantially comprising cells, e.g., red blood cells.

Accordingly, in one aspect, a microfluidic device is disclosed, said microfluidic device comprising: a base comprising a microfluidic channel, wherein the microfluidic channel approximates a pattern selected from the group consisting of a substantially circular spiral, a substantially elliptical spiral, a substantially serpentine pattern, and a substantially straight channel,
wherein the microfluidic channel has a cross-section that is square, rectangular, circular, triangular, polygonal, or elliptical, and is about 0.5 mm to about 10 mm deep and about 0.5 mm to about 10 mm wide.

In another aspect, a method of separating whole blood into at least a fraction comprising plasma serum and a fraction comprising cells and other solid matter, said method comprising: introducing whole blood into the microfluidic channel of the microfluidic cassette described herein, holding the whole blood in the microfluidic channel for an amount of time necessary to effectuate substantial sedimentation, resulting in a top layer that comprises plasma serum and a bottom layer that comprises cells and other solid matter; and
removing the layers by pumping or vacuuming, wherein the top layer moves with a higher velocity than the bottom layer, and the top layer comprising the plasma serum moves past the bottom layer to an exit port.

Without being bound by theory, the method described herein involves laminar flow of the plasma serum past the sedimented cells of the whole blood. Accordingly, the present device and method does not necessarily have to be microfluidic, so long as sedimentation of a solid material can be efficiently performed followed by the removal of the less dense liquid, relative to the more dense solid material, by laminar flow. Accordingly, although the device and method of using same described herein has been described as microfluidic, it is understood by the person skilled in the art that the device can be a fluidic device, and the channels can be wider and deeper than described for the microfluidic channel This permits the use of this technology as part of a medical device when other materials are to be separated.

It is also noted that the device and method of using same has been described to separate red blood cells and other solid matter from plasma serum. That said, the device and method of using same described herein can be used to separate prokaryotic or eukaryotic cells from the red blood cells and other solid matter in the blood sample, using the same microfluidic device and method of using same wherein sedimentation with subsequent laminar flow is relied on. Using the device and method of using said device described herein, following sedimentation in the device, the collected plasm serum can comprise prokaryotic or eukaryotic cells because blood cells sediment much more quickly than the prokaryotic or eukaryotic cells. The prokaryotic or eukaryotic cells therefore still suspended in the plasma serum after sedimentation can therefore be removed with the plasma serum fraction. This aspect of the invention is important when the presence of bacteria and other prokaryotic or eukaryotic cells in blood needs to be detected or identified using an assay or other experimental technique.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments w47ithin the spirit and scope of the claims hereafter set forth.

What is claimed is:

1. A microfluidic cassette comprising:
a base comprising a microfluidic channel, wherein the microfluidic channel approximates a pattern selected from the group consisting of a substantially circular spiral, a substantially elliptical spiral, a substantially serpentine pattern, and a substantially straight channel,
wherein the microfluidic channel has a cross-section that is square, rectangular, circular, triangular, polygonal, or elliptical, and is about 0.5 mm to about 10 mm deep and about 0.5 mm to about 10 mm wide.

2. The microfluidic cassette of claim 1, wherein the microfluidic channel is in or on a base.

3. The microfluidic cassette of claim 1, wherein the microfluidic channel resides in a single plane.

4. The microfluidic cassette of claim 1, further comprising an entry port at one end, and an exit port at another end, of the microfluidic channel

5. The microfluidic cassette of claim 4, wherein the entry port and the exit port each comprise a connector for connecting tubes thereto.

6. The microfluidic cassette of claim 5, wherein a first tube is used to introduce a fluid to the microfluidic cassette, and a second tube is used to remove a fluid from the microfluidic cassette.

7. A method of separating whole blood into at least a fraction comprising plasma serum and a fraction comprising cells and other solid matter, said method comprising:
introducing whole blood into the microfluidic channel of the microfluidic cassette of claim 1, holding the whole blood in the microfluidic channel for an amount of time necessary to effectuate substantial sedimentation, resulting in a top layer that comprises plasma serum and a bottom layer that comprises cells and other solid matter; and
removing the layers by pumping or vacuuming, wherein the top layer moves with a higher velocity than the bottom layer, and the top layer comprising the plasma serum moves past the bottom layer to an exit port.

8. The method of claim 7, further comprising collecting an initial fraction of the top layer removed from the microfluidic device, wherein the initial fraction comprises plasma serum.

9. The method of claim 7, wherein the microfluidic channel is in or on a base.

10. The method of claim 7, wherein the microfluidic channel resides in a single plane.

11. The method of claim 7, further comprising an entry port at one end, and an exit port at another end, of the microfluidic channel

12. The method of claim 11, wherein the entry port and the exit port each comprise a connector for connecting tubes thereto.

13. The method of claim 8, further comprising a second purification step utilizing affinity binding to remove any remaining cells and other solid matter from the plasma serum.

14. The method of claim 8, further comprising directing the plasma serum to subsequent reaction or monitoring assays.

15. The method of claim 7, wherein the method avoids the use of centrifuges or anything requiring centrifugal force and filtration units or any other separation devices requiring membranes.

16. The method of claim 7, wherein the plasma serum is substantially devoid of hemoglobin, lysing chemicals, and anti-coagulants.

17. The method of claim 7, wherein the removing comprises the use of a syringe to push or pull the plasma serum out of the microfluidic channel while the cells and other solid matter remain in the channel

18. The method of claim 7, wherein the plasma serum is highly pure plasma serum.

19. The method of claim 7, wherein the plasma serum comprises prokaryotic or eukaryotic cells.
